(19) United States
(12) Patent Application Publication
(10) Pub. No.: US 2024/0341829 A1
(43) Pub. Date: Oct. 17, 2024

(54) SYSTEM AND METHOD FOR DETERMINING AND DISPLAYING CRYOABLATION INFORMATION

(71) Applicant: Boston Scientific Scimed, Inc., Maple Grove, MN (US)

(72) Inventor: Chadi Harmouche, Quebec (CA)

(21) Appl. No.: 18/634,617

(22) Filed: Apr. 12, 2024

Related U.S. Application Data
(60) Provisional application No. 63/496,083, filed on Apr. 14, 2023.

(51) Int. Cl. A61B 18/02; A61B 18/00

(52) U.S. Cl. CPC A61B 18/02; A61B 2018/0022; A61B 2018/00351; A61B 2018/00577; A61B 2018/00648; A61B 2018/00666; A61B 2018/00791; A61B 2018/00839; A61B 2018/0212

(57) ABSTRACT

A cryogenic balloon catheter system for use in an ablation procedure, the cryogenic balloon catheter system comprising a balloon catheter including a shaft, an expandable balloon attached to a distal portion of the shaft, a plurality of sensors each configured to sense an ablation parameter during an ablation phase of the ablation procedure and to generate a sensor output based on the sensed ablation parameter a fluid source operatively coupled to the expandable balloon and configured to deliver a cryogenic fluid to the expandable balloon, a controller configured to receive the sensor output and calculate a plurality of ablation metrics and to generate a plurality of ablation quality indicator outputs, and a graphical display operatively coupled to the controller and configured to display a plurality of graphical ablation quality indicators each based on a respective one of the ablation quality indicator outputs.

### CROSS REFERENCE TO RELATED APPLICATION

This application claims priority to and the benefit of U.S. Provisional Application No. 63/496,083, filed Apr. 14, 2023, which is incorporated by reference herein in its entirety.

### TECHNICAL FIELD

The present disclosure relates to medical devices and methods for performing cryoablation procedures using a cryoablation catheter. More specifically, the disclosure relates to devices and methods for determining and displaying ablation quality information.

### BACKGROUND

Cardiac arrhythmias involve an abnormality in the electrical conduction of the heart and are a leading cause of stroke, heart disease, and sudden cardiac death. Treatment options for patients with arrhythmias include medications and use of medical devices, including implantable devices and catheter ablation of cardiac tissue. Catheter ablation involves delivering ablative energy to tissue inside the heart to block aberrant electrical activity from depolarizing heart muscle cells out of synchrony with the heart's normal conduction pattern. An ablation procedure may be performed by positioning the tip of an energy delivery catheter adjacent to targeted tissue in or near the heart.

The energy delivery component of the system is typically at or near the most distal (i.e., the furthest from the user or operator) portion of the catheter, and often at the tip of the catheter. Various forms of energy can be used to ablate diseased heart tissue. These can include, for example, radio frequency (RF), cryogenics, ultrasound, electrical fields, and laser energy. During a cryoablation procedure, with the aid of a guide wire, the distal tip of the catheter is positioned adjacent to targeted cardiac tissue, at which time energy is delivered to create tissue necrosis, rendering the ablated tissue incapable of conducting electrical signals. The dose of the energy delivered is an important factor in increasing the likelihood that the treated tissue is rendered permanently incapable of conduction.

Atrial fibrillation (AF) is one of the common arrhythmias treated using catheter ablation. In the earliest stages of the disease, paroxysmal AF, the treatment strategy may involve isolating the pulmonary veins from the left atrial chamber. One catheter procedure to treat AF is known as “balloon cryotherapy,” which offers ease of use, shorter procedure times and improved patient outcomes.

A goal of balloon cryotherapy is to isolate one or more pulmonary veins of the patient by creating circumferential transmural lesions around an ostium of the pulmonary vein being treated. During balloon cryotherapy, one or more cryogenic balloons are placed in the left atrium and positioned against the ostium of the pulmonary vein to occlude blood flow from the pulmonary veins into the left atrium. With the cryogenic balloons appropriately positioned to occlude the targeted tissue, a cryogenic fluid (e.g., nitrous oxide) is delivered under pressure to an interior of the one or more cryogenic balloons. The cryogenic fluid causes necrosis of the targeted tissue, thereby rendering the ablated tissue incapable of conducting electrical signals.

### SUMMARY

In Example 1, a cryogenic balloon catheter system for use in an ablation procedure, the cryogenic balloon catheter system comprising a balloon catheter, a plurality of sensors, a fluid source, a controller, and a graphical display. The balloon catheter includes a shaft, and an expandable balloon attached to a distal portion of the shaft. The plurality of sensors each are configured to sense an ablation parameter during an ablation phase of the ablation procedure and to generate a sensor output based on the sensed ablation parameter. The fluid source is operatively coupled to the expandable balloon and is configured to deliver a cryogenic fluid to the expandable balloon. The controller is configured to receive the sensor output from each of the plurality of sensors and calculate a plurality of ablation metrics each based on one or more of the sensed ablation parameters, and to generate a plurality of ablation quality indicator outputs each based on a comparison of one of the calculated ablation metrics and a respective target ablation metric. The graphical display is operatively coupled to the controller and is configured to display a plurality of graphical ablation quality indicators having a visual appearance based on a respective one of the ablation quality indicator outputs.

In Example 2, the cryogenic balloon catheter system of claim 1, wherein the controller includes a timer configured to track a time elapsed during the ablation phase.

In Example 3, the cryogenic balloon catheter system of claim 2, wherein the plurality of sensors includes a temperature sensor configured to generate a temperature sensor output indicative of a sensed expandable balloon temperature.

In Example 4, the cryogenic balloon catheter system of claim 3, wherein the plurality of ablation metrics includes an expandable balloon temperature at a prescribed time of the ablation phase, and wherein the target ablation metric is a target sensed expandable balloon temperature at the prescribed time.

In Example 5, the cryogenic balloon catheter system of claim 4, wherein the plurality of ablation metrics includes a lowest expandable balloon temperature during the ablation phase, and wherein the target ablation metric is a target lowest sensed expandable balloon temperature during the ablation phase.

In Example 6, the cryogenic balloon catheter system of any of claims 3-5, wherein the plurality of sensors includes an electrical sensor configured to sense cardiac electrical signals and generate an electrical sensor output based thereon, and wherein the controller is configured to detect a time-to-isolation based on the electrical sensor output and the tracked time elapsed during the ablation phase of the ablation procedure.

In Example 7, the cryogenic balloon catheter system of claim 6, wherein the plurality of ablation metrics includes a time-to-isolation, and wherein the target ablation metric is a prescribed time-to-isolation.

In Example 8, the cryogenic balloon catheter system of any of claims 1-7, wherein the visual appearance of each of the ablation quality indicators varies based on the respective comparison of the calculated ablation metric and the target ablation metric.

In Example 9, the cryogenic balloon catheter of any of claims 1-7, wherein the visual appearance of each of the ablation quality indicators provides a visual indication to the user of whether the calculated ablation metric meets the target ablation metric.

In Example 10, the cryogenic balloon catheter system of any of claims 1-8, wherein each of the target ablation metrics is pre-programmed in the controller.

In Example 11, the cryogenic balloon catheter system of any of claims 1-8, wherein one or more of the target ablation metrics is selected by a user via a user interface operatively coupled to the graphical display.

In Example 12, the cryogenic balloon catheter system of any of claims 3-10, wherein the graphical display is configured to display a graphical representation of the sensed expandable balloon temperature over a duration of the ablation phase.

In Example 13, the cryogenic balloon catheter system of claim 11, wherein the graphical display is configured to display, based on an output from the controller, a target ablation metric graph based on one or more of the target ablation metrics over time for the duration of the ablation phase.

In Example 14, the cryogenic balloon catheter system of claim 12, wherein the target ablation metric graph is based on the one or more target ablation metrics and a pre-determined tolerance.

In Example 15, the cryogenic balloon catheter system of claim 13, wherein the graphical display is configured to superimpose the graphical representation of the sensed expandable balloon temperature over the target ablation metric graph.

In Example 16, a cryogenic balloon catheter system for use in an ablation procedure, the cryogenic balloon catheter system comprising a balloon catheter, a plurality of sensors, a controller and a graphical display. The balloon catheter includes a shaft, and an expandable balloon attached to a distal portion of the shaft. The plurality of sensors each are configured to sense an ablation parameter during an ablation phase of the ablation procedure and to generate a sensor output based on the sensed ablation parameter. The controller is configured to receive the sensor output from each of the plurality of sensors and calculate a plurality of ablation metrics each based on one or more of the sensed ablation parameters, and to generate a plurality of ablation quality indicator outputs each based on a comparison of one of the calculated ablation metrics and a respective target ablation metric. The graphical display is operatively coupled to the controller and is configured to display a plurality of graphical ablation quality indicators having a visual appearance based on a respective one of the ablation quality indicator outputs.

In Example 17, the cryogenic balloon catheter system of claim 16, wherein the plurality of sensors includes a temperature sensor configured to generate a temperature sensor output indicative of a sensed expandable balloon temperature, and the plurality of ablation metrics includes an expandable balloon temperature at a prescribed time of the ablation phase, and wherein the target ablation metric is a target sensed expandable balloon temperature at the prescribed time.

In Example 18, the cryogenic balloon catheter system of claim 16, wherein the plurality of ablation metrics includes a lowest expandable balloon temperature during the ablation phase, and wherein the target ablation metric is a target lowest sensed expandable balloon temperature during the ablation phase.

In Example 19, the cryogenic balloon catheter system of claim 16, wherein the plurality of sensors includes an electrical sensor configured to sense cardiac electrical signals and generate an electrical sensor output based thereon, and wherein the controller is configured to detect a time-to-isolation based on the electrical sensor output and the tracked time elapsed during the ablation phase of the ablation procedure, and further wherein the plurality of ablation metrics includes a time-to-isolation, and wherein the target ablation metric is a prescribed time-to-isolation.

In Example 20, the cryogenic balloon catheter system of claim 16, wherein the visual appearance of each of the ablation quality indicators varies based on the respective comparison of the calculated ablation metric and the target ablation metric.

In Example 21, the cryogenic balloon catheter of claim 20, wherein the visual appearance of each of the ablation quality indicators provides a visual indication to the user of whether the calculated ablation metric meets the target ablation metric.

In Example 22, the cryogenic balloon catheter system of claim 21, wherein each of the target ablation metrics is pre-programmed in the controller.

In Example 23, the cryogenic balloon catheter system of claim 21, wherein one or more of the target ablation metrics is selected by a user via a user interface operatively coupled to the graphical display.

In Example 24, the cryogenic balloon catheter system of claim 16, wherein the graphical display is configured to display, based on an output from the controller, a target ablation metric graph based on one or more of the target ablation metrics over time for the duration of the ablation phase.

In Example 25, the cryogenic balloon catheter system of claim 24, wherein the target ablation metric graph is based on the one or more target ablation metrics and a pre-determined tolerance.

In Example 26, a control system for cryogenic balloon catheter system for use in an ablation procedure, the control system comprising a controller and a graphical display. The controller is configured to, receive a plurality of sensor outputs each based on a sensed ablation parameter, calculate a plurality of ablation metrics each based on one or more of the sensed ablation parameters, and generate a plurality of ablation quality indicator outputs each based on a comparison of one of the calculated ablation metrics and a respective target ablation metric. The graphical display is operatively coupled to the controller and is configured to display a plurality of graphical ablation quality indicators having a visual appearance based on a respective one of the ablation quality indicator outputs.

In Example 27, the control system of claim 26, wherein the plurality of sensors includes a temperature sensor configured to generate a temperature sensor output indicative of a sensed expandable balloon temperature, and the plurality of ablation metrics includes an expandable balloon temperature at a prescribed time of the ablation phase, and wherein the target ablation metric is a target sensed expandable balloon temperature at the prescribed time.

In Example 28, the control system of claim 26, wherein the plurality of ablation metrics includes a lowest expandable balloon temperature during the ablation phase, and wherein the target ablation metric is a target lowest sensed expandable balloon temperature during the ablation phase.

In Example 29, the control system of claim 26, wherein the plurality of sensors includes an electrical sensor configured to sense cardiac electrical signals and generate an electrical sensor output based thereon, and wherein the controller is configured to detect a time-to-isolation based on the electrical sensor output and the tracked time elapsed during the ablation phase of the ablation procedure, and further wherein the plurality of ablation metrics includes a time-to-isolation, and wherein the target ablation metric is a prescribed time-to-isolation.

In Example 30, the control system of claim 26, further comprising a user interface configured to receive, from a user, selected values for each target ablation metric.

In Example 31, the control system of claim 26, wherein the graphical display is configured to display, based on an output from the controller, a target ablation metric graph based on one or more of the target ablation metrics over time for the duration of the ablation phase.

In Example 32, the control system of claim 31, wherein the target ablation metric graph is based on the one or more target ablation metrics and a pre-determined tolerance.

In Example 33, a method of displaying cryoablation information during an ablation phase of a cryoablation procedure, the method comprising receiving, by a controller of a control system of a balloon catheter system, a plurality of sensor outputs each based on a sensed ablation parameter, calculating, by the controller, a plurality of ablation metrics each based on one or more of the sensed ablation parameters, generating, by the controller, a plurality of ablation quality indicator outputs each based on a comparison of one of the calculated ablation metrics and a respective target ablation metric, and displaying, on a graphical display of the balloon catheter system, a plurality of graphical ablation quality indicators having a visual appearance based on a respective one of the ablation quality indicator outputs.

In Example 34, the method of claim 33, further comprising receiving, by the controller, user-selected values for each respective target ablation metric.

In Example 35, the method of claim 33, further comprising displaying, by the graphical display and based on an output from the controller, a target ablation metric graph based on one or more of the target ablation metrics over time for the duration of the ablation phase.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic side view illustration of a patient and of a cryogenic balloon catheter system, including a cryogenic balloon catheter assembly and a control and display system according to embodiments of the present invention.

FIG. 2 is a schematic view illustration of a portion of a patient's heart and a portion of the cryogenic balloon catheter system, including a pressure sensor, according to embodiments of the present invention.

FIG. 3 is a simplified flow diagram illustrating one exemplary method that may be carried out by the cryogenic balloon catheter system of FIG. 1 during an ablation phase of a cryoablation procedure, according to an embodiment of the present disclosure.

FIG. 4 is an illustration of an exemplary ablation metric setup screen that may be accessible by a user and visible on the graphical display of the cryogenic balloon catheter system of FIG. 1, according to embodiments of the present disclosure.

FIGS. 5A-5C are exemplary graphical display windows that can be incorporated into the graphical display of the cryogenic balloon catheter system of FIG. 1, according to embodiments of the present disclosure.

FIGS. 6A-6D illustrate portions of exemplary graphical display screens implementing various embodiments of the present disclosure to provide the user with clinically-useful ablation metric information.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, and/or dimensions are provided for selected elements. Those skilled in the art will recognize that many of the noted examples have a variety of suitable alternatives.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

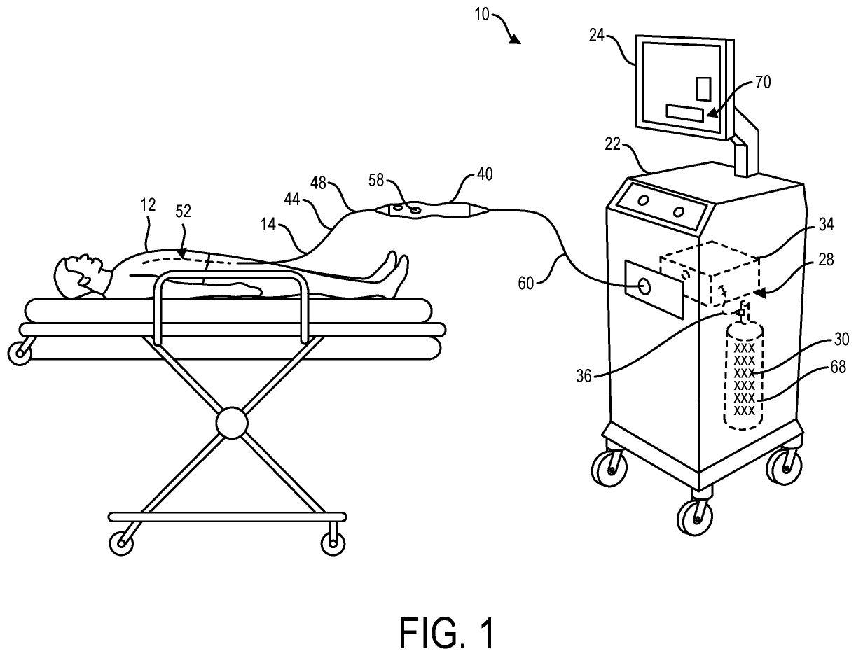

FIG. 1 is a simplified schematic side view illustration of an embodiment of a cryogenic balloon catheter system 10 for use with a patient 12. Although the design of the cryogenic balloon catheter system 10 can be varied depending on the particular clinical needs of the patient 12, in the illustrated embodiment, the cryogenic balloon catheter system 10 can include one or more of a balloon catheter 14, a control console 22, a graphical display 24, and a fluid control system 28 (illustrated in phantom and disposed within the control console 22 in FIG. 1). In the illustrated embodiment, the fluid control system 28 includes a fluid source 30 and a fluid control arrangement 34. In the various embodiments, the fluid control system 28 can include various conduits, valves and instrumentation configured to supply and withdraw a fluid to the active elements on the balloon catheter 14 as will be described in greater detail elsewhere herein. In the illustrated embodiment, the fluid source 30 is operably connected to the fluid control arrangement 34 by a conduit 36 (which may be in the form of a hose or tubing) configured to transfer fluid contained within the fluid source 30 to components making up the fluid control arrangement 34.

As further shown, the balloon catheter 14 includes a handle assembly 40, and a shaft 44 having a proximal end portion 48 connected to the handle assembly 40, and a distal end portion 52, shown disposed within the patient 12 in FIG. 1. As will be appreciated, the handle assembly 40 can include various components, such as the control element 58 in FIG. 1, that the user can manipulate to operate the balloon catheter 14. Also, in the particular embodiment illustrated in FIG. 1, an umbilical 60 operatively connects the handle assembly 40 and the active components of the balloon catheter 14 to the control console 22.

In various embodiments, the system 10 may also include additional components or alternative approaches to operatively connect the balloon catheter 14 to the control console 22. That is, the particular means of operatively connecting these elements is not critical the present disclosure, and so any suitable means can be employed.

It is understood that although FIG. 1 illustrates the structures of the cryogenic balloon catheter system 10 in a particular position, sequence and/or order, these structures can be located in any suitably different position, sequence and/or order than that illustrated in FIG. 1. It is also understood that the cryogenic balloon catheter system 10 can include fewer or additional components than those specifically illustrated and described herein.

In various embodiments, the fluid control system 28 is configured to monitor and control various processes of the ablation procedures performed with the cryogenic balloon catheter system 10. More specifically, the fluid control system 28 can monitor and control release and/or retrieval of a cooling fluid 68, e.g., a cryogenic fluid (shown schematically contained within the fluid source 30 in FIG. 1), to the balloon catheter 14, e.g., via fluid injection and fluid exhaust lines (not shown, but which may be disposed within the umbilical 60. The fluid control system 28 can also control various structures that are responsible for maintaining and/or adjusting a flow rate and/or pressure of the cryogenic fluid 68 that is released to the balloon catheter 14 during the cryoablation procedure. In such embodiments, the cryogenic balloon catheter system 10 delivers ablative energy in the form of cryogenic fluid 68 to cardiac tissue of the patient 12 to create tissue necrosis, rendering the ablated tissue incapable of conducting electrical signals. Additionally, in various embodiments, the fluid control system 28 can control activation and/or deactivation of one or more other processes of the balloon catheter 14.

The fluid control system includes, among other things, a controller comprising, without limitation, a processor (which may be implemented in a general-purpose or special purpose computing device), memory, input/output components, and a power supply. Any number of additional components, different components, and/or combinations of components may also be included in the controller.

In embodiments, memory includes computer-readable media in the form of volatile and/or nonvolatile memory and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In embodiments, the memory stores computer-executable instructions for causing the controller to implement aspects of embodiments of system components discussed herein and/or to perform aspects of embodiments of methods and procedures discussed herein. Computer-executable instructions may include, for example, computer code, machine-useable instructions, and the like such as, for example, program components capable of being executed by one or more processors associated with the controller. Program components may be programmed using any number of different programming environments, including various languages, development kits, frameworks, and/or the like. Some or all of the functionality contemplated herein may also, or alternatively, be implemented in hardware and/or firmware.

In embodiments, the controller of the fluid control system 28 can receive data and/or other information (hereinafter sometimes referred to as “sensor output”) from various sensors within the cryogenic balloon catheter system 10. In some embodiments, the fluid control system 28 can receive, monitor, assimilate and/or integrate the sensor output, and/or any other data or information received from any structure within the cryogenic balloon catheter system 10 in order to control the operation of the balloon catheter system 10. In embodiments, the sensors include temperature sensors positioned on the balloon catheter 14 and configured to sense and generate an output indicative of temperature within the expandable cryoballoon of the balloon catheter 14 and/or the external environment, e.g., the cardiac chamber in which the balloon catheter 14 is disposed. Additionally, the controller of the control system 28 can receive sensor output from electrical sensors, e.g., electrodes on the balloon catheter 14 or ancillary devices (as described in greater detail below) configured to generate an electrical sensor output indicative of intrinsic electrical activity of the cardiac tissue. Such electrical sensor outputs can be processed by the control system to monitor parameters indicative of the status of the ablation procedure.

In embodiments, the control system 28 further includes a timing system including a timer configured to track a time elapsed during the various phases of a cryoablation procedure. As will be explained in greater detail below, the tracked time is a component of various clinically-useful ablation metric calculations made by the controller of the control system 28, which can assist the user in assessing the efficacy of the cryoablation procedure and the likelihood that an effective conduction block has been achieved.

As shown in FIG. 1, in certain embodiments, the fluid control system 28 can be positioned substantially within the control console 22. Alternatively, at least a portion of the fluid control system 28 can be positioned in one or more other locations within the cryogenic balloon catheter system 10, e.g., within the handle assembly 40.

The fluid source 16 contains the cryogenic fluid 68, which is delivered to and from the balloon catheter 14 with or without input from the fluid control system 28 during a cryoablation procedure. Once the ablation procedure has been initiated, the cryogenic fluid 68 can be delivered and the resulting gas, after a phase change, can be withdrawn from the balloon catheter 14, and can either be vented or otherwise discarded as exhaust. Additionally, the type of cryogenic fluid 68 that is used during the cryoablation procedure can vary. In one non-exclusive embodiment, the cryogenic fluid 68 within the fluid source 16 may be liquid nitrous oxide. However, any other suitable cryogenic fluid 68 can be used. For example, in one non-exclusive alternative embodiment, the cryogenic fluid 68 can include liquid nitrogen.

The design of the balloon catheter 14 can be varied to suit the specific design requirements of the cryogenic balloon catheter system 10. As shown, the balloon catheter 14 is inserted into the body of the patient 12 during the cryoablation procedure. The handle assembly 40 can be handled and used by the operator to operate, position and control the balloon catheter 14. The design and specific features of the handle assembly 40 can vary to suit the design requirements of the cryogenic balloon catheter system 10. In the embodiment illustrated in FIG. 1, the handle assembly 40 is separate from, but in electrical and/or fluid communication with the fluid control system 28, the fluid source 16, and the graphical display 24. In some embodiments, the handle assembly 40 can integrate and/or include at least a portion of the fluid control system 28 within an interior of the handle assembly 40. It is understood that the handle assembly 40 can include fewer or additional components than those specifically illustrated and described herein. Additionally, in certain embodiments, the handle assembly 40 can include circuitry (not shown in FIG. 1) that can include at least a portion of the fluid control system 28. Alternatively, the circuitry can transmit electrical signals such as the sensor output, or otherwise provide data to the fluid control system 28 as described herein. In one embodiment, the circuitry can include a printed circuit board having one or more integrated circuits, or any other suitable circuitry.

Still further, in certain embodiments, the handle assembly 40 can be used by the operator to initiate and/or terminate the cryoablation process, e.g., to start the flow of the cryogenic fluid 68 to the balloon catheter 14 in order to ablate certain targeted heart tissue of the patient 12.

In the embodiment illustrated in FIG. 1, the control console 22 includes at least a portion of the fluid control system 28, the fluid source 16, and the graphical display 24. However, in alternative embodiments, the control console 22 can contain additional structures not shown or described herein. Still alternatively, the control console 22 may not include various structures that are illustrated within the control console 22 in FIG. 1.

In various embodiments, the graphical display 24 is electrically connected to the controller of the fluid control system 28. Additionally, the graphical display 24 provides the operator of the cryogenic balloon catheter system 10 with information that can be used before, during and after the cryoablation procedure. For example, the graphical display 24 can provide the operator with information based on the sensor output, and any other relevant information that can be used before, during and after the cryoablation procedure. The specifics of the graphical display 24 can vary depending upon the design requirements of the cryogenic balloon catheter system 10, or the specific needs, specifications and/or desires of the operator.

In one embodiment, the graphical display 24 can provide static visual data and/or information to the operator via various frames or other representations (depicted as element 70 in FIG. 1). In addition, or in the alternative, the graphical display 24 can provide dynamic visual data and/or information to the operator, such as video data or any other data that changes over time, e.g., during an ablation procedure. Further, in various embodiments, the graphical display 24 can include one or more colors, different sizes, varying brightness, etc., that may act as alerts to the operator. Additionally, or in the alternative, the graphical display 24 can provide audio data or information to the operator.

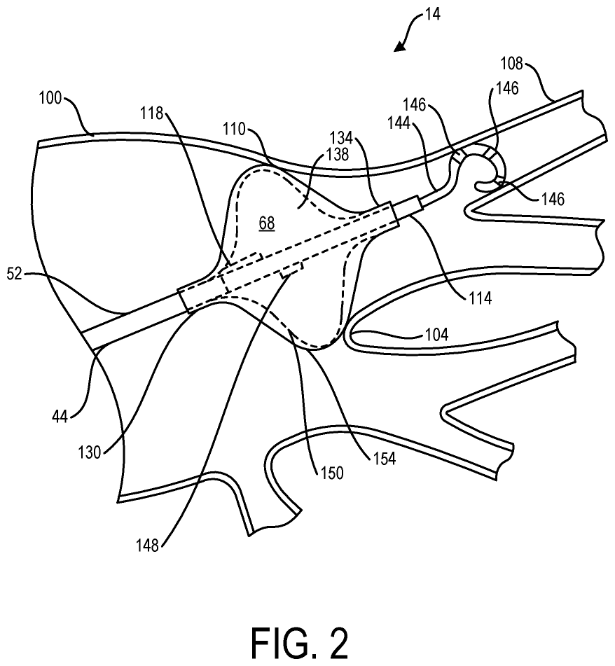

FIG. 2 is a schematic illustration of the distal end portion 52 of the balloon catheter 14 positioned within a selected anatomical region of the patient 12, in this case, a left atrium 100 adjacent to an ostium 104 of a pulmonary vein 108, such as when the system 10 is used in a pulmonary vein isolation (PVI) procedure to terminate an atrial fibrillation. In the illustrated embodiment, the balloon catheter 14 includes an expandable balloon 110, a guidewire lumen 114 and an injection tube 118. As shown, the balloon 110 has a proximal end 130 and an opposite distal end 134, and defines an internal space 138 that creates a cryo-chamber during a cryoablation procedure. In the illustrated embodiment, the proximal end 130 of the balloon 110 is attached to the distal end portion 52 of the shaft 44, and the distal end 134 of the balloon 110 is attached to the guidewire lumen 114 near the distal end thereof. In the illustrated embodiment, the injection tube 118 is disposed within and extends from the shaft 44, and terminates within and is open to the internal space 138. The injection tube 118 is operable to deliver the cryogenic fluid 68 to the internal space 138.

Although not shown in FIG. 2, the balloon catheter 14 also includes an exhaust lumen within the shaft 44 and open to the internal space 138. The exhaust lumen is operable to facilitate evacuation of the cryogenic fluid 68 from the internal space 138, and in some embodiments, may optionally be used to facilitate rapid inflation of the balloon 110.

In various embodiments, the guidewire lumen may be slidable relative to the shaft 44 to facilitate expansion and subsequent collapse of the balloon 110 in use. However, the particular construction of the balloon 110 and guidewire lumen 114 is not critical to the present disclosure, and so other configurations may be used within the scope of the various embodiments.

For illustration purposes, a mapping wire 144 is shown extending through and beyond the guidewire lumen 114 and into the pulmonary vein 108. As shown, the mapping wire 144 includes a plurality of electrical sensors 146 (e.g., electrodes) which are operatively coupled to the controller of the control system 28 (FIG. 1) and are configured to sense cardiac electrical signals generated within target cardiac tissue, e.g., the tissues proximate the pulmonary veins. Additionally, the balloon catheter 14 includes a temperature sensor 148 (e.g., a thermocouple assembly, a thermistor, or the like) positioned within the internal space 138 and operatively coupled to the controller of the control system 28 (FIG. 1). The temperature sensor 148 is configured to sense the temperature within the internal space 138 during the various phases of a cryoablation procedure, and to generate a temperature sensor output that is used by the controller to, among other things, calculate selected ablation metrics during the ablation phase of the cryoablation procedure, as will be explained in greater detail below.

In the embodiment of FIG. 2, the balloon 110 is a dual-balloon construction including an inner balloon 150 and an outer balloon 154. The balloons 150, 154 are configured such that the inner balloon 150 receives the cryogenic fluid 68 (illustrated in FIG. 1), and the outer balloon 154 surrounds the inner balloon 150. The outer balloon 154 acts as part of a safety system to capture the cryogenic fluid 68 in the event of a leak from the inner balloon 150. It is understood that the balloon catheter 14 can include other structures as well. However, for the sake of clarity, these other structures have been omitted from the figures. Additionally, it is further appreciated that in some alternative embodiments, the balloon catheter 14 includes only a single balloon.

In the embodiment illustrated in FIG. 2, the balloon catheter 14 is positioned within the left atrium 100 of the patient 12. The instrument 144 and guidewire lumen 114 are inserted into a pulmonary vein 108 of the patient 12, and the catheter shaft 44 and the balloons 150, 154 are moved along the guidewire 144 and/or the guidewire lumen 114 to be positioned near an ostium 104 of the pulmonary vein 108.

During use, the inner balloon 150 can be partially or fully inflated so that at least a portion of the inner balloon 150 expands against at least a portion of the outer balloon 154. Once the inner balloon 150 is sufficiently inflated, an outer surface of the outer balloon 154 can then be positioned to abut and/or substantially form a seal with the ostium 104 of the pulmonary vein 108 to be treated.

The inner balloon 150 and the outer balloon 154 can be formed from any suitable materials. For example, in some embodiments, the inner balloon 150 can be formed from a sturdy material to better inhibit leaks of the cryogenic fluid 68 that is received therein, and the outer balloon 154 can be made from a relatively compliant material to ensure better contact and positioning between the outer balloon 154 and the pulmonary vein 108.

Referring to FIGS. 1 and 2 together, the various embodiments of the cryogenic balloon catheter system 10 can provide the user, via the graphical display 24, with visual indications of clinically-useful ablation metrics to assist the user in assessing the likely efficacy of a cryoablation procedure and to make subsequent clinical decisions based on the aforementioned assessment. In particular, the controller is configured to calculate, based on one or more sensor outputs (e.g., the sensor output from the temperature sensor 148) one or more ablation metrics, compare each calculated ablation metric with a corresponding target ablation metric, and cause the graphical display to display a graphical ablation quality indicator based on the aforementioned comparison. The ablation quality indicator provides the user with a clear, easy to read indication whether, for each selected ablation metric, the target ablation metric has been achieved during the ablation phase of a cryoablation procedure.

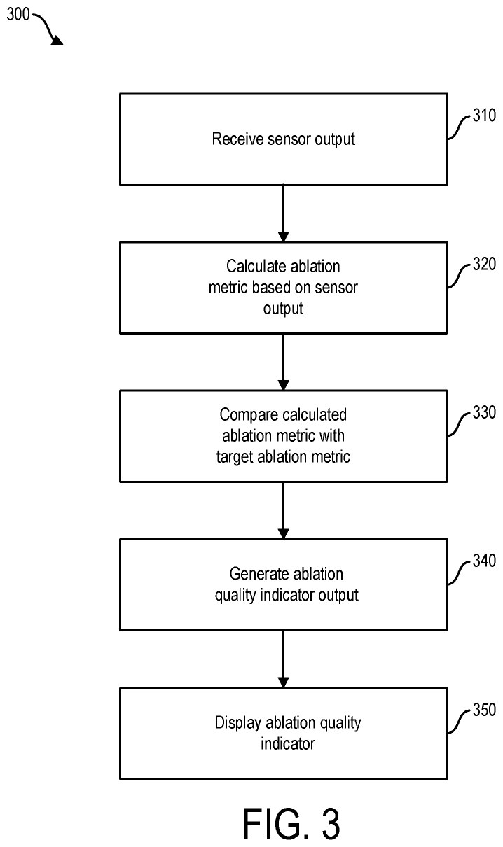

FIG. 3 is a simplified flow diagram illustrating one exemplary method 300 that may be carried out by the cryogenic balloon catheter system 10 during a cryoablation procedure, particularly during the ablation phase of a cryoablation procedure, according to an embodiment of the present disclosure. As shown, at Block 310, the controller receives sensor outputs from one or more sensors (e.g., the temperature sensor 148 or the electrical sensors 146 shown in FIG. 2), wherein each sensor output is indicative of a sensed ablation parameter such as temperature within the internal space 138 defined by the balloon 110 (in the case of the temperature sensor 148) and/or cardiac electrical activity (in the case of the electrical sensors 146).

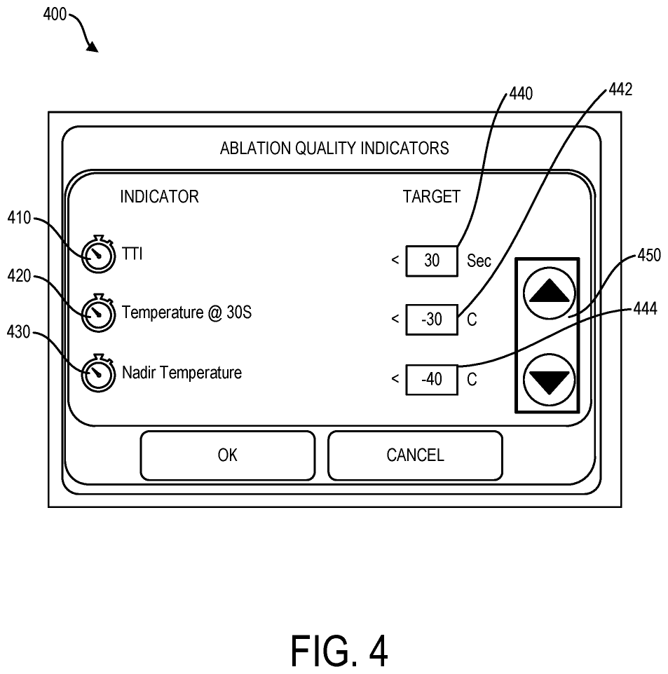

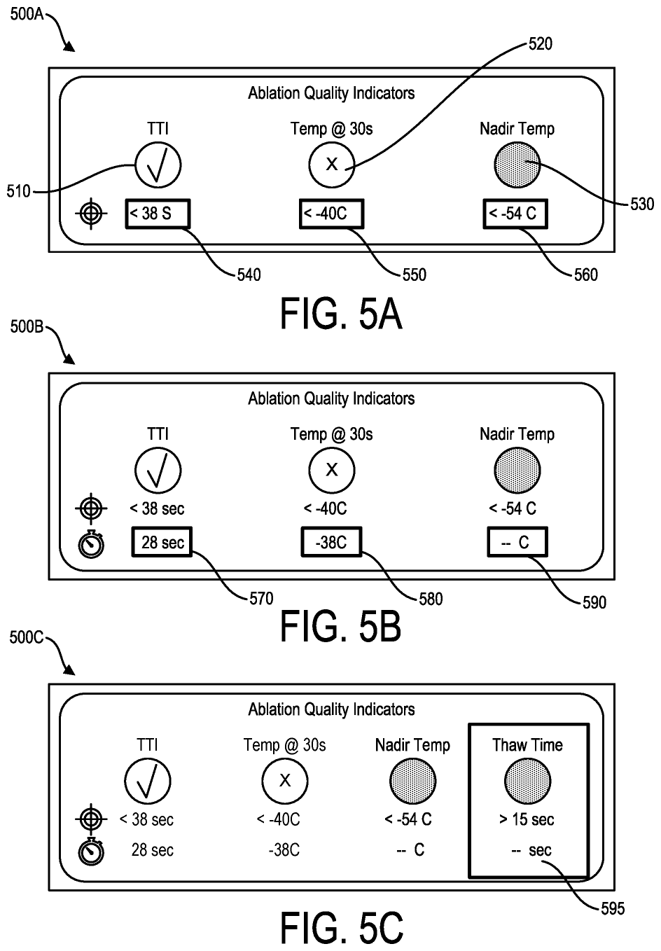

At Block 320, the controller calculates one or more ablation metrics each based on the received sensor output(s). Specific exemplary ablation metrics that may be calculated are described in greater detail below in connection with FIG. 4 and FIGS. 5A-5D. Then, at Block 330, the controller performs a comparison of each calculated ablation metric with a target ablation metric, and generates an ablation quality indicator output based on each comparison performed. Each ablation quality indicator output comprises an electrical signal that is responsive to the results of the aforementioned comparison. Then, at Block 340, upon receipt of each ablation quality indicator output from the controller, the graphical display 24 (see FIG. 1) displays a graphical ablation quality indicator based on corresponding to each ablation metric. In embodiments, the visual appearance of each ablation quality indicator may be based on the received ablation quality indicator outputs.

In embodiments, the particular ablation metrics utilized for a given ablation procedure may be pre-programmed into the memory of the controller, or alternatively, may be selected by the user via a user interface from a number of available options. Similarly, the target ablation metrics may be pre-programmed, default values, or may be user-selectable via a user interface that is part of or operatively coupled to the control system 28 or graphical display 24, based on particular preferences of the user.

FIG. 4 is an illustration of an exemplary ablation metric setup screen 400 that may be accessible by a user and visible on the graphical display for defining particular ablation metrics to be calculated and displayed, and also for selecting particular target ablation metrics, and FIGS. 5A-5C are exemplary graphical display windows that can be incorporated into the graphical display for visualization by the user. Referring to FIGS. 1-2 as well as FIG. 4, in the exemplary embodiment of FIG. 4, the setup screen 400 displays three available ablation metrics-a TTI (time-to-isolation) metric 410, a temperature-at-time metric 420, and a nadir temperature metric 430. As further shown, the setup screen further includes target ablation metric setting fields 440, 442, 444 corresponding to each of the ablation metrics 410, 420, 430, as well as selection buttons 450 that can be manipulated by the user to adjust each target ablation metric according to his or her own preferences. In some embodiments, each of the target ablation metrics 410, 420, 430 may be pre-programmed at prescribed values, e.g., based on empirical data from prior cryoablation procedures, and may be adjusted by the user according to the user's preferences.

In the illustrated embodiment, the TTI metric 410 is based on a detected “time-to-isolation” during a given ablation phase In embodiments, the time-to-isolation may refer to the point in time during the ablation phase when cardiac electrical signals can no longer be sensed by the electrical sensors 146 on the mapping wire 144 (see FIG. 2). In the particular embodiment, the target ablation metric for the TTI metric 410 has been set to “<30 Sec,” and the controller will compare the actual detected time-to-isolation based on the outputs from the electrical sensors 146 and the associated time stamp from the timer of the control system 28, with the target TTI metric 410, and will generate the ablation quality indicator output based on this comparison, as described in greater detail in connection with FIGS. 5A-5C.

The temperature-at-time metric 420 is based on the sensed balloon catheter temperature (based in turn on the corresponding temperature sensor output) at the prescribed time during the ablation phase. In the exemplary embodiment, the selected prescribed time is 30 seconds, and the target ablation metric is a sensed balloon temperature (i.e., the temperature within the internal space 138, see FIG. 2) of <minus 30 degrees Celsius at that prescribed time. In the particular embodiment, the prescribed time is fixed (i.e., 30 seconds) but in other embodiments both the target temperature and the prescribed time may be either pre-programmed, user-adjustable, or both. The nadir temperature metric 430 corresponds to the lowest sensed balloon temperature over the entire duration of the ablation phase. In the illustrated embodiment, the target nadir temperature metric is a sensed lowest temperature of below minus 40 degrees Celsius.

FIG. 5A illustrates a window 500A of the graphical display 24 (see FIG. 1) corresponding to the ablation metrics 410, 420 and 430 illustrated in the setup screen 400 of FIG. 4. Accordingly, as shown in FIG. FIG. 5A, the window 500A includes ablation quality indicators 510, 520 and 530 corresponding, respectively, to the TTI metric 410, the temperature-at-time metric 420 and the nadir temperature metric 430. The window 500A further includes target ablation metric settings 540, 550, 560 for each of the metrics 410, 420, 430. As can be seen in FIG. 5A, the appearance on the graphical display 24 of each ablation quality indicator varies as a function of whether the target ablation metric has been achieved. Thus for example, the TTI metric indicator 510 is annotated with a check mark indicating that the target TTI metric (in this case, less than 38 seconds) has been achieved (meaning, the actual time-to-isolation was detected by the controller as occurring earlier than 38 seconds after the start of the ablation phase). In contrast, the visual appearance for the temperature-at-time metric has an appearance indicating to the user that the target ablation metric (in this case, a sensed temperature of less than minus 40 degrees Celsius 30 seconds after the start of the ablation phase) was not achieved. Finally, the appearance of the nadir temperature indicator 530 has an appearance indicating that the comparison between the calculated ablation metric and the target ablation metric has not yet been made, for example, because the ablation phase has not yet been completed and so the lowest sensed temperature over the entire duration of the ablation phase cannot yet be determined.

FIG. 5B illustrates an exemplary alternative window 500B that is substantially the same as the window 500A, with the addition of fields 570, 580, 590 indicating the actual calculated or determined TTI metric, temperature-at-time metric, and nadir temperature metric. FIG. 5C illustrates an exemplary window 500C that is substantially identical to the window 500B, with the addition of an additional “Thaw Time” ablation metric 595 and corresponding target and actual calculated values.

It will be appreciated by the skilled artisan that the particular visual appearances depicted for the ablation quality indicators shown in FIGS. 5A-5C are exemplary only. In embodiments, other visual appearances may be utilized and selected to provide the user with a clear indication of the status of each calculated ablation metric relative to the corresponding target ablation metric. For example, in embodiments, a color coding system may be utilized, e.g., green when the calculated ablation metric meets or exceeds the target value, red when the calculated ablation metric does not meet the target value, and grey or black when the controller is still processing the ablation metric data or where the relevant time period (e.g., the entire duration of the ablation procedure with respect to the nadir temperature metric) has not elapsed.

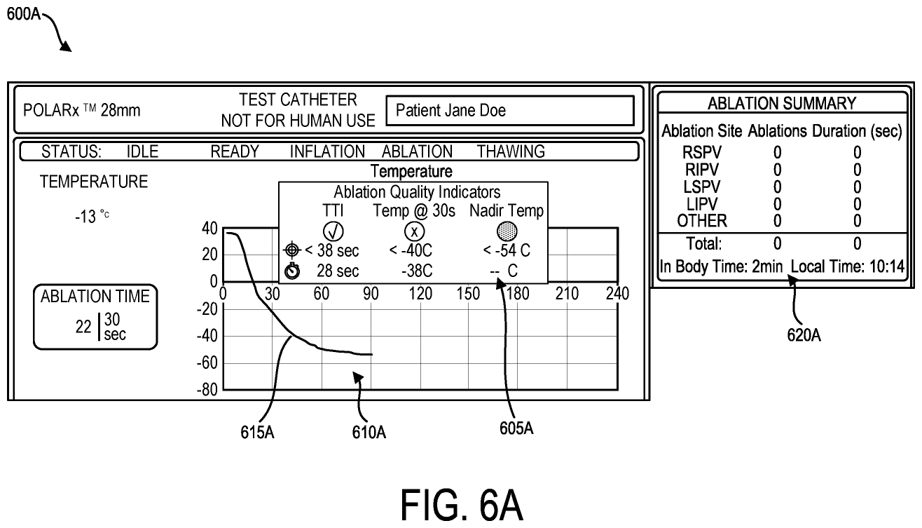

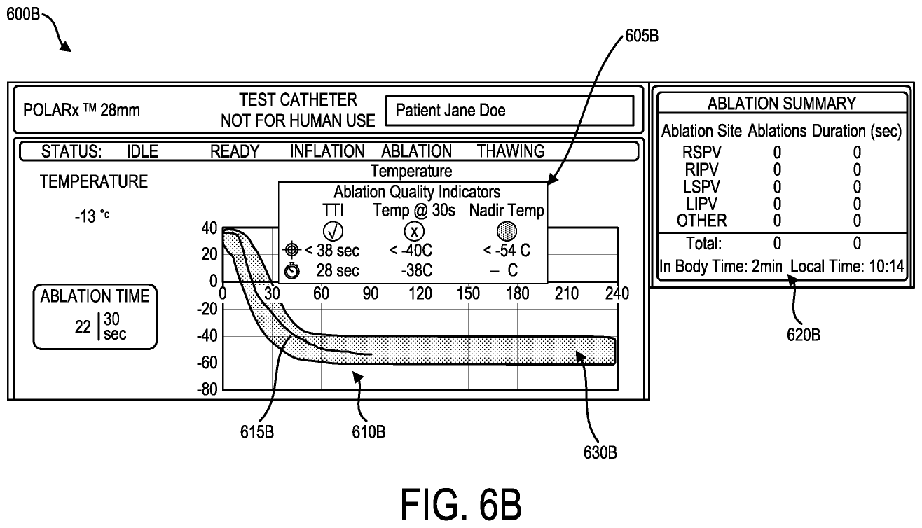

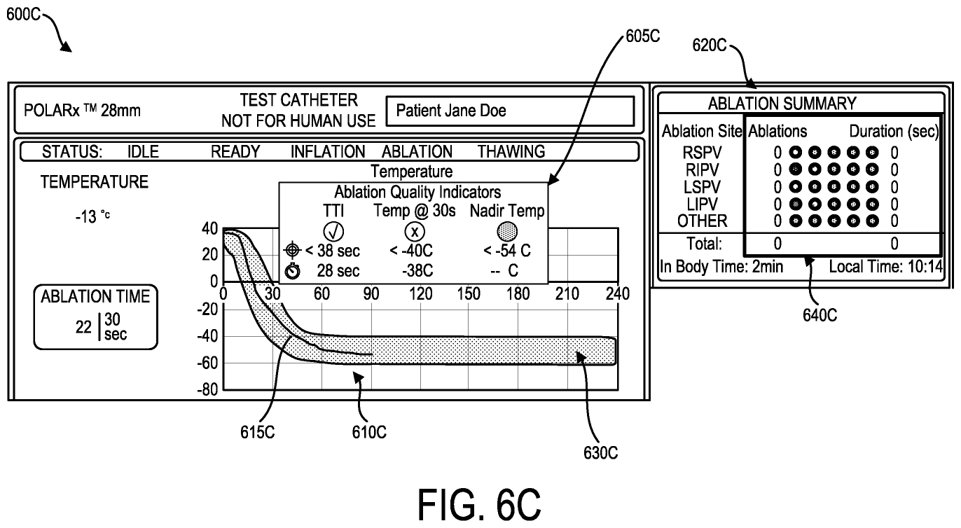

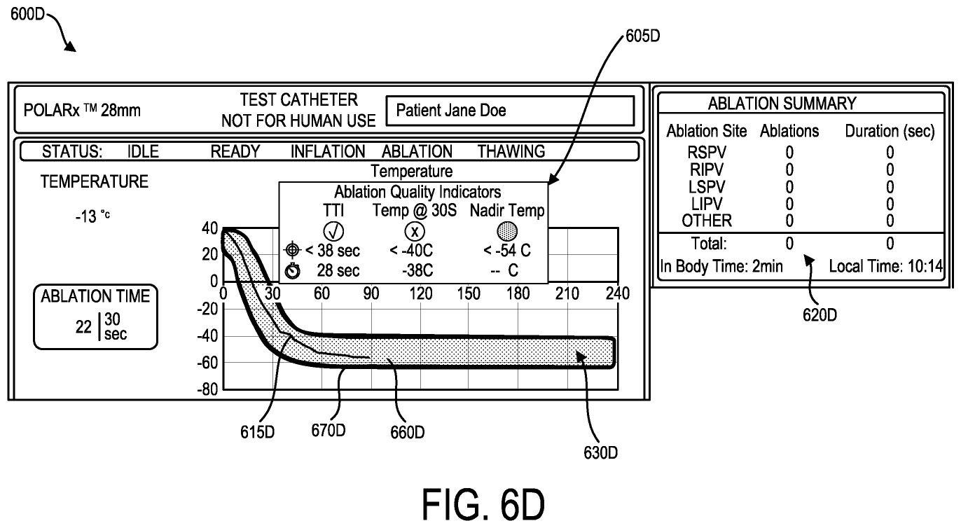

FIGS. 6A-6D illustrate portions of exemplary graphical display screens 600A, 600B, 600C and 600D, respectively, implementing various embodiments of the present disclosure to provide the user with clinically-useful ablation metric information. As shown in FIG. 6A, the display screen 600A includes, among other things, an ablation quality indicator window 605A that corresponds to the window 500B shown in FIG. 5B, as well as an ablation progress chart 610A that depicts a temperature trace 615A of the actual sensed balloon temperature (vertical axis) over time (horizontal axis). Additionally, in the illustrated embodiment, and ablation summary window 620A that can provide a single-view summary of clinically-useful information to the user in respect of ablations performed at each of the pulmonary veins (i.e., the right superior pulmonary vein (RSPV), the right inferior pulmonary vein RIPV, the left superior pulmonary vein LSPV and the left inferior pulmonary vein LIPV.

As shown in FIG. 6B, the display screen 600B is substantially identical to the display screen 600A, and includes an ablation quality indicator window 605B, an ablation progress chart 610B depicting a sensed temperature trace 615B, and an ablation summary window 620B. As further shown, the display screen 600B further includes a target ablation metric graph 630B, and the temperature trace 615B is superimposed over the target ablation metric graph 630B. In embodiments, the target ablation metric graph 630B is derived from one or more of the selected target ablation metrics, and provides a visual indication of the progress of the ablation phase relative to the target ablation metrics. In one embodiment, the geometry of the target ablation graph 630B may be generated based on one or more of the selected target ablation metrics and a pre-determined tolerance or deviation from the target metric at any given time point during the ablation phase. For example, in one embodiment, the center point of the target ablation graph at a particular time point may correspond to a predicted sensed temperature value at that time point based on the selected target temperature-at-time metric and/or the selected target nadir temperature metric, and the width of the target ablation metric graph may be based on a suitable tolerance or deviation from the predicted target temperature. In such an embodiment, by superimposing the actual sensed temperature trace 615A over the target ablation metric graph 630B, the user is provided with a visual indication of the likely effectiveness of the particular ablation phase as it progresses. For example, if in a given procedure the temperature trace 615A deviates from within the upper or lower edges of the target ablation metric graph 630B, the user may conclude that adjustments to various ablation parameters may be warranted. Of course, this particular example is exemplary only.

As shown in FIG. 6C, the display screen 600C is substantially identical to the display screen 600B, and includes an ablation quality indicator window 605C, an ablation progress chart 610C depicting a sensed temperature trace 615C and a target ablation metric graph 630C. The display screen 600C includes an alternative ablation summary window 620C comprising multiple composite ablation quality indicators 640C for each applied ablation process at each of the aforementioned locations. In embodiments, each of the individual composite ablation indicators corresponds to one or more ablation metrics calculated during each respective ablation phase and the results of the comparison of these ablation metrics with the corresponding target ablation metric. The composite ablation quality indicators may be annotated to provide a visual indication of the extent to which the target ablation metrics were met in a given ablation phase. For example, in an ablation in which all target ablation metrics were met, the corresponding composite ablation quality indicator may be colored green or have some annotation providing a clear indication to the user. Conversely, in an ablation phase in which none of the target ablation metrics were achieved, the corresponding composite ablation indicator may be colored red or annotated similarly to indicate to provide a clear indication to the user of the potential effectiveness of the applied ablation. Still other color coding (e.g., yellow) or similar annotations may be applied where some, but not all, of the target ablation metrics were achieved. The ablation summary window 620C thus provides an elegant, easy to visualize indication of the potential effectiveness of multiple ablation cycles over multiple target locations.

As shown in FIG. 6D, the display screen 600D is substantially identical to the display screen 600B, and includes an ablation quality indicator window 605D, an ablation progress chart 610D depicting a sensed temperature trace 615D, an ablation summary window 620D, a target ablation metric graph 630D. However, in the embodiment of FIG. 6D, the target ablation metric graph includes an inner region 660D and an outer region 670D. The inner and outer regions 660D and 670D may be derived in the same manner but using different acceptable tolerances or deviations from the predicted ablation metric value at each point in time during the ablation phase.

While the specific embodiments described herein utilize certain ablation metrics, i.e., the TTI metric, the temperature-at-time metric, and the nadir temperature metric, the subject matter of the present disclosure is adaptable to accept any clinically-useful ablation metrics whether now known or later developed. Non-limiting examples of additional useful ablation metrics may include the number of ablations required to achieve pulmonary vein isolation per patient or per pulmonary vein, the temperature at initial pulmonary vein isolation at each pulmonary vein, the rate of sensed temperature decrease over a prescribed time period (e.g., 30 seconds) and the time required to reach a prescribed sensed temperature (e.g., minus 40 degrees Celsius or minus 50 degrees Celsius). Thus, the subject matter of the present disclosure is in no way limited to the specific ablation metrics specifically described herein.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A cryogenic balloon catheter system for use in an ablation procedure, the cryogenic balloon catheter system comprising:
a balloon catheter including a shaft, and an expandable balloon attached to a distal portion of the shaft;
a plurality of sensors each configured to sense an ablation parameter during an ablation phase of the ablation procedure and to generate a sensor output based on the sensed ablation parameter;
a controller configured to receive the sensor output from each of the plurality of sensors and calculate a plurality of ablation metrics each based on one or more of the sensed ablation parameters, and to generate a plurality of ablation quality indicator outputs each based on a comparison of one of the calculated ablation metrics and a respective target ablation metric; and
a graphical display operatively coupled to the controller and configured to display a plurality of graphical ablation quality indicators having a visual appearance based on a respective one of the ablation quality indicator outputs.

2. The cryogenic balloon catheter system of claim 1, wherein the plurality of sensors includes a temperature sensor configured to generate a temperature sensor output indicative of a sensed expandable balloon temperature, and the plurality of ablation metrics includes an expandable balloon temperature at a prescribed time of the ablation phase, and wherein the target ablation metric is a target sensed expandable balloon temperature at the prescribed time.

3. The cryogenic balloon catheter system of claim 1, wherein the plurality of ablation metrics includes a lowest expandable balloon temperature during the ablation phase, and wherein the target ablation metric is a target lowest sensed expandable balloon temperature during the ablation phase.

4. The cryogenic balloon catheter system of claim 1, wherein the plurality of sensors includes an electrical sensor configured to sense cardiac electrical signals and generate an electrical sensor output based thereon, and wherein the controller is configured to detect a time-to-isolation based on the electrical sensor output and the tracked time elapsed during the ablation phase of the ablation procedure, and further wherein the plurality of ablation metrics includes a time-to-isolation, and wherein the target ablation metric is a prescribed time-to-isolation.

5. The cryogenic balloon catheter system of claim 1, wherein the visual appearance of each of the ablation quality indicators varies based on the respective comparison of the calculated ablation metric and the target ablation metric.

6. The cryogenic balloon catheter of claim 5, wherein the visual appearance of each of the ablation quality indicators provides a visual indication to the user of whether the calculated ablation metric meets the target ablation metric.

7. The cryogenic balloon catheter system of claim 6, wherein each of the target ablation metrics is pre-programmed in the controller.

8. The cryogenic balloon catheter system of claim 6, wherein one or more of the target ablation metrics is selected by a user via a user interface operatively coupled to the graphical display.

9. The cryogenic balloon catheter system of claim 1, wherein the graphical display is configured to display, based on an output from the controller, a target ablation metric graph based on one or more of the target ablation metrics over time for the duration of the ablation phase.

10. The cryogenic balloon catheter system of claim 9, wherein the target ablation metric graph is based on the one or more target ablation metrics and a pre-determined tolerance.

11. A control system for cryogenic balloon catheter system for use in an ablation procedure, the control system comprising:
a controller configured to:
receive a plurality of sensor outputs each based on a sensed ablation parameter;
calculate a plurality of ablation metrics each based on one or more of the sensed ablation parameters, and;
generate a plurality of ablation quality indicator outputs each based on a comparison of one of the calculated ablation metrics and a respective target ablation metric; and
a graphical display operatively coupled to the controller and configured to display a plurality of graphical ablation quality indicators having a visual appearance based on a respective one of the ablation quality indicator outputs.

12. The control system of claim 11, wherein the plurality of sensors includes a temperature sensor configured to generate a temperature sensor output indicative of a sensed expandable balloon temperature, and the plurality of ablation metrics includes an expandable balloon temperature at a prescribed time of the ablation phase, and wherein the target ablation metric is a target sensed expandable balloon temperature at the prescribed time.

13. The control system of claim 11, wherein the plurality of ablation metrics includes a lowest expandable balloon temperature during the ablation phase, and wherein the target ablation metric is a target lowest sensed expandable balloon temperature during the ablation phase.

14. The control system of claim 11, wherein the plurality of sensors includes an electrical sensor configured to sense cardiac electrical signals and generate an electrical sensor output based thereon, and wherein the controller is configured to detect a time-to-isolation based on the electrical sensor output and the tracked time elapsed during the ablation phase of the ablation procedure, and further wherein the plurality of ablation metrics includes a time-to-isolation, and wherein the target ablation metric is a prescribed time-to-isolation.

15. The control system of claim 11, further comprising a user interface configured to receive, from a user, selected values for each target ablation metric.

16. The control system of claim 11, wherein the graphical display is configured to display, based on an output from the controller, a target ablation metric graph based on one or more of the target ablation metrics over time for the duration of the ablation phase.

17. The control system of claim 16, wherein the target ablation metric graph is based on the one or more target ablation metrics and a pre-determined tolerance.

18. A method of displaying cryoablation information during an ablation phase of a cryoablation procedure, the method comprising:
receiving, by a controller of a control system of a balloon catheter system, a plurality of sensor outputs each based on a sensed ablation parameter;
calculating, by the controller, a plurality of ablation metrics each based on one or more of the sensed ablation parameters,
generating, by the controller, a plurality of ablation quality indicator outputs each based on a comparison of one of the calculated ablation metrics and a respective target ablation metric; and
displaying, on a graphical display of the balloon catheter system, a plurality of graphical ablation quality indicators having a visual appearance based on a respective one of the ablation quality indicator outputs.

19. The method of claim 18, further comprising receiving, by the controller, user-selected values for each respective target ablation metric.

20. The method of claim 18, further comprising displaying, by the graphical display and based on an output from the controller, a target ablation metric graph based on one or more of the target ablation metrics over time for the duration of the ablation phase.
